SEQUENCE LISTING (1 sequence)

SEQ ID NO: 1 - protein, 14 aa, Artificial sequence
  Synthetic peptide
XAEELDSRAGXPQL

(19) United States
(12) Patent Application Publication
(10) Pub. No.: US 2012/0115865 A1
(43) Pub. Date: May 10, 2012

(54) New Salts of an Indole Derivative and Their Use in Medicine

(76) Inventors: Anna-Lena Berg, Sodertalje (SE); Ratan Bhat, Sodertalje (SE); Tesfai Sebhatu, Sodertalje (SE); Erica Stahle, Sodertalje (SE)

(21) Appl. No.: 12/280,270

(22) PCT Filed: Jan. 31, 2007
(86) PCT No.: PCT/SE07/00089

§ 371 Date: Oct. 30, 2008

Related U.S. Application Data
(60) Provisional application No. 60/777,348, filed on Feb. 28, 2006.

(51) Int. Cl. A61K 31/5377; A61P 25/18; A61P 25/28; A61P 25/00; A61P 25/16; A61P 3/10; A61P 19/08; A61P 19/10; A61P 29/00; A61P 17/14; C07D 413/14; A61P 25/24

(52) U.S. Cl. 514/235.2; 544/124

(57) ABSTRACT

The present invention relates to new pharmaceutically acceptable salts of 2-hydroxy-3-[5-(morpholin-4-yl-methyl)pyridin-2-yl]1H-indole-5-carbonitrile, processes for their preparations, pharmaceutical formulations containing said salts and to the use of said active salts in therapy, and particularly to GSK3 related disorders.

### FIELD OF THE INVENTION

The present invention relates to new pharmaceutically acceptable salts of 2-hydroxy-3-[5-(morpholin-4-ylmethyl)pyridin-2-yl]1H-indole-5-carbonitrile, a process for their preparations, pharmaceutical formulations containing said salts and to the use of said active salts in therapy.

### BACKGROUND OF THE INVENTION

2-Hydroxy-3-[5-(morpholin-4-ylmethyl)pyridin-2-yl]1H-indole-5-carbonitrile as a free base and the hydrochloride salt thereof are described in WO 03/082853. This compound is useful because it possess pharmacological activity by showing inhibiting effect on GSK3 (WO 03/082853). This compound could be used to treat Alzheimer disease, dementias, chronic and acute neurodegenerative diseases, bipolar disorders, schizophrenia, diabetes, hair loss, bone-related disorders and all the listed disorders described in WO 03/082853, which hereby are incorporated into this specification by reference.

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3(3) or Tau phosphorylating kinase selectively phosphorylates the microtubule associated protein tau in neurons at sites that are hyperphosphorylated in AD brains. Hyperphosphorylated tau has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK313 phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Accumulation of amyloid-β is an early event in AD. GSK Tg mice show increased levels of amyloid-β in brain. Also, PDAPP mice fed with Lithium show decreased amyloid-β levels in hippocampus and decreased amyloid plaque area (Su et al., Biochemistry 2004, 43:6899-6908). Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as cognitive disorders, Alzheimer's Disease, Parkinson's Disease, amyotrophic lateral sclerosis, Huntington's Disease and HIV dementia and traumatic brain injury; and as in ischemic stroke. Lithium was neuroprotective in inhibiting apoptosis in cells and in the brain at doses that resulted in the inhibition of GSK3β. Thus GSK3Jβ inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996; Gould et al., Neuropsychopharmacology, 1:32-8, 2004). GSK3 inhibitor has been shown to reduce immobilisation time in forced swim test, a model to assess on depressive behavior (O'Brien et al., J Neurosci 2004, 24:66791-6798) GSK3 has been associated with a polymorphism found in bipolar II disorder (Szczepankiewicz et al., Neuropsychobiology. 2006; 53(1):51-6). Inhibition of GSK3β may therefore be of therapeutic relevance in the treatment of BD as well as in AD patients that have affective disorders.

### Schizophrenia

Accumulating evidence implicates abnormal activity of GSK3 in mood disorders and schizophrenia. GSK3 is involved in signal transduction cascades of multiple cellular processes, particularly during neural development. Kozlovsky et al (Am J Psychiatry 2000 May; 157(5):831-3) found that GSK313 levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)). Atypical antipsychotics such as olanzapine, clozapine, quetiapine, and ziprasidone, inhibits GSK3 by increasing ser9 phosphorylation suggesting that antipsychotics may exert their beneficial effects via GSK3 inhibition (Rosborough et al., Int J Neuropsychopharmacol, 4:1-13 2006).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2):263-71). Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51:2903-2910; Ring et al., Diabetes 2003, 52:588-595). GSK3 inhibition may therefore be of therapeutic relevance in the treatment of Type I and Type II diabetes and diabetic neuropathy.

### Alopecia

GSK3 phosphorylates and degrades β-catenin. β-catenin is an effector of the pathway for keratonin synthesis. β-catenin stabilisation may be lead to increase hair development. Mice expressing a stabilised p-catenin by mutation of sites phosphorylated by GSK3 undergo a process resembling de novo hair morphogenesis (Gat et al., Cell 1998 Nov. 25; 95 (5):605-14)). The new follicles formed sebaceous glands and dermal papilla, normally established only in embryogenesis. Thus GSK3 inhibition may offer treatment for baldness.

### Bone-Related Disorders and Conditions

GSK3 inhibitors could be used for treatment of bone-related disorders or other conditions, which involves a need for new and increased bone formation. Remodeling of the skeleton is a continuous process, controlled by systemic hormones such as parathyroid hormone (PTH), local factors (e.g. prostaglandin E₂), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

The use of GSK3 inhibitors in primary and secondary osteoporosis, where primary osteoporosis includes postmenaupausal osteoporosis and senile osteoporosis in both men and women, and secondary osteoporosis includes cortison induced osteoporosis, as well as any other type of induced secondary osteoporosis. In addition to this, GSK3 inhibitors may also be used in treatments of myeloma. The GSK3 inhibitors may be administered locally or systemically, in different formulation regimes, to treat these conditions.

### Inflammatory Disease

The discovery that GSK3 inhibitors provide anti-inflammatory effects has raised the possibility of using GSK3 inhibitors for therapeutic intervention in inflammatory diseases. (Martin et al., Nat 1 mmol 2005, 6:777-784; rev. in Jope et al., Neurochem Res 2006, Aug. 30). Inflammation is a common feature of a broad range of conditions including Alzheimer's Disease and mood disorders.

### DETAILED DESCRIPTION OF THE INVENTION

The object of the present invention is to provide new salts of the compound of 2-hydroxy-3-[5-(morpholin-4-ylmethyl)pyridin-2-yl]1H-indole-5-carbonitrile (compound (I))

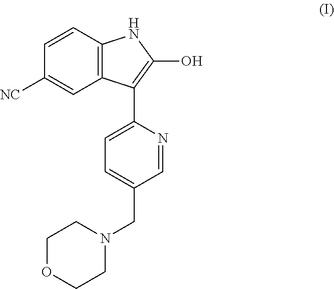

having a selective inhibiting effect at GSK3, a good bioavailability, a good solubility and a low hygroscopicity which making them suitable to be formulated into pharmaceutical formulations. These new salts are the mesylate, esylate, edisylate, phosphate, fumarate and maleate of 2-hydroxy-3-[5-(morpholin-4-ylmethyl)pyridin-2-yl]1H-indole-5-carbonitrile.

A further aspect of the invention relates to
2-hydroxy-3-[5-(morpholin-4-ylmethyl)pyridin-2-yl]1H-indole-5-carbonitrile mesylate,
2-hydroxy-3-[5-(morpholin-4-ylmethyl)pyridin-2-yl]1H-indole-5-carbonitrile esylate,
2-hydroxy-3-[5-(morpholin-4-ylmethyl)pyridin-2-yl]1H-indole-5-carbonitrile edisylate,
2-hydroxy-3-[5-(morpholin-4-ylmethyl)pyridin-2-yl]1H-indole-5-carbonitrile phosphate,
2-hydroxy-3-[5-(morpholin-4-ylmethyl)pyridin-2-yl]1H-indole-5-carbonitrile fumarate and 2-hydroxy-3-[5-(morpholin-4-ylmethyl)pyridin-2-yl]1H-indole-5-carbonitrile maleate.

These salts of the compound of formula (I) according to the present invention have been found to show an improved chemical stability over the hydrochloride salt of 2-hydroxy-3-[5-(morpholin-4-ylmethyl)pyridin-2-yl]1H-indole-5-carbonitrile prepared as described in WO 03/082853 which makes said salts particularly suitable to be formulated into pharmaceutical formulations.

In the formulation of pharmaceutical formulations, it is important for the pharmaceutically acceptable compound (the active drug compound) to be in a form in which it can be conveniently handled and processed. This is of importance, not only from the point of view is of obtaining a commercially viable manufacturing process, but also from the point of view of subsequent manufacture of pharmaceutical formulations comprising the active drug compound.

Chemical stability, solid state stability, and “shelf-life” of the active ingredients are also very important factors. The drug compound and formulations containing it should be capable of being effectively stored over appreciable periods of time, without exhibiting a significant change in physico-chemical characteristics of the active component, e.g. its chemical composition, density, hygroscopicity and solubility.

The term “chemical stability” means that the compound can be stored in an isolated form, or in the form of a formulation in which it is provided in admixture with pharmaceutically acceptable carriers, diluents or adjuvants (e.g., in an oral dosage form, such as tablet, capsule, etc.), under normal storage conditions, with little or no chemical degradation or decomposition.

Thus, in the manufacture of commercially viable and pharmaceutically acceptable drug formulations it is important, wherever possible, to provide the drug compound in a substantially crystalline and stable form.

As used herein, the term “substantially crystalline” means at least about 50% crystalline and ranging up to 100% crystalline. The present invention provides 2-hydroxy-3-[5-(morpholin-4-ylmethyl)pyridin-2-yl]1H-indole-5-carbonitrile salts that is at least about 50% crystalline, at least about 60% crystalline, at least about 70% crystalline, at least about 80% crystalline, at least about 90% crystalline, at least about 95% crystalline, at least about 98% crystalline, or about 100% crystalline in form.

### Pharmaceutical Formulations

According to one aspect of the present invention there is provided a pharmaceutical formulation comprising the mesylate, esylate, edisylate, phosphate, fumarate or maleate salt of the compound (I), 2-hydroxy-3-[5-(morpholin-4-ylmethyl)pyridin-2-yl]1H-indole-5-carbonitrile, for use in the prevention and/or treatment of conditions associated with glycogen synthase kinase-3.

The formulation may be in a form suitable for oral administration, for example as a tablet, for parenteral injection as a sterile solution or suspension, for local administration in a body cavity or in a bone cavity, for example as a sterile injection solution or suspension.

In general the above formulation may be prepared in a conventional manner using pharmaceutically carriers or diluents. Suitable daily doses of the salt of the compound of formula (I) in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

For the veterinary use the amounts of different components, the dosage form and the dose of the medicament may vary and will depend on various factors as for example the individual requirement of the animal treated.

A pharmaceutically acceptable salt of the compound of formula (I), can be used on its own but will usually be administered in the form of a pharmaceutical formulation in which the to formula (I) compound salt (active ingredient) is in association with pharmaceutically acceptable diluents, excipients or inert carrier. Dependent on the mode of administration, the pharmaceutical formulation may comprise from 0.05 to 99% w (percent by weight), for example from 0.10 to 50% w, of active ingredient, all percentages by weight being based on total composition.

A diluent or carrier includes water, aqueous poly(ethylene glycol), magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A formulation of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical formulation of the invention, which comprises mixing a pharmaceutically acceptable salt of the compound of formula (I), as hereinbefore defined, with pharmaceutically acceptable diluents, excipients or inert carriers.

An example of a pharmaceutical formulation of the invention is an injectable solution comprising a pharmaceutically acceptable salt of the compound of formula (I), as hereinbefore defined, and sterile water, and, if necessary, either sodium hydroxide or hydrochloric acid to bring the pH of the final formulation to about pH 5, and optionally a surfactant to aid dissolution.

A liquid solution comprising 2-hydroxy-3-[5-(morpholin-4-ylmethyl)pyridin-2-yl]1H-indole-5-carbonitrile mesylate, esylate, edisylate, phosphate, fumarate or maleate 5.0% mg/mL dissolved in pure water to 100%.

### Medical Uses

It has been found that the new salts 2-hydroxy-3-[5-(morpholin-4-ylmethyl)pyridin-2-yl]1H-indole-5-carbonitrile mesylate, esylate, edisylate, phosphate, fumarate or maleate defined in the present invention, are well suited for inhibiting glycogen synthase kinase-3 (GSK3). Accordingly, said compounds of the present invention are expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with cognitive disorders and predemented states, especially dementia, Alzheimer's Disease (AD), Cognitive Deficit in Schizophrenia (CDS), Mild Cognitive Impairment (MCI), Age-Associated Memory Impairment (AAMI), Age-Related Cognitive Decline (ARCD) and Cognitive Impairement No Dementia (CIND), diseases associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration (CBD), traumatic brain injury (TBI) and dementia pugilistica.

One embodiment of the invention relates to the prevention and/or treatment of Alzheimer's Disease, especially the use in the delay of the disease progression of Alzheimer's Disease.

Other conditions are selected from the group consisting of Down's syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND, Creurtfeld-Jacob's disease and prion diseases.

Other conditions are selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other conditions are selected from the group consisting of Type I diabetes, Type II diabetes, diabetic neuropathy, alopecia and inflammatory diseases.

One embodiment of the invention relates to the prevention and/or treatment of bone-related disorders in mammals.

Another aspect of the invention is directed to the use of 2-hydroxy-3-[5-(morpholin-4-ylmethyl)pyridin-2-yl]1H-indole-5-carbonitrile mesylate, esylate, edisylate, phosphate, fumarate and maleate in the prevention and/or treatment of to treat osteoporosis in mammals.

One aspect of the invention is directed to the use of 2-hydroxy-3-[5-(morpholin-4-ylmethyl)pyridin-2-yl]1H-indole-5-carbonitrile mesylate, esylate, edisylate, phosphate, fumarate and maleate to promote and/or increase bone formation in mammals.

One aspect of the invention is directed to the use of 2-hydroxy-3-[5-(morpholin-4-ylmethyl)pyridin-2-yl]1H-indole-5-carbonitrile mesylate, esylate, edisylate, phosphate, fumarate and maleate to increase bone mineral density in mammals.

Another aspect of the invention is directed to the use of 2-hydroxy-3-[5-(morpholin-4-ylmethyl)pyridin-2-yl]1H-indole-5-carbonitrile mesylate, esylate, edisylate, phosphate, fumarate and maleate to reduce the rate of fracture and/or increase the rate of fracture healing in mammals.

Another aspect of the invention is directed to the use of 2-hydroxy-3-[5-(morpholin-4-ylmethyl)pyridin-2-yl]1H-indole-5-carbonitrile mesylate, esylate, edisylate, phosphate, fumarate and maleate to increase cancellous bone formation and/or new bone formation in mammals.

The dose required for the therapeutic or preventive treatment of a particular disease will necessarily be varied depending on the host treated, the route of administration and the severity of the illness being treated.

The present invention relates also to the use of 2-hydroxy-3-[5-(morpholin-4-ylmethyl)pyridin-2-yl]1H-indole-5-carbonitrile mesylate, esylate, edisylate, phosphate, fumarate and maleate in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, including man in need of such treatment and/or prevention a therapeutically effective amount of the 2-hydroxy-3-[5-(morpholin-4-ylmethyl)pyridin-2-yl]1H-indole-5-carbonitrile mesylate, esylate, edisylate, phosphate, fumarate and maleate.

### Method of Salt Formation

The formation of the desired salt of the compound of formula (I), 2-hydroxy-3-[5-(morpholin-4-ylmethyl)pyridin-2-yl]1H-indole-5-carbonitrile mesylate, esylate, edisylate, phosphate, fumarate or maleate, may be prepared by mixing 2-hydroxy-3-[5-(morpholin-4-ylmethyl)pyridin-2-yl]1H-indole-5-carbonitrile with the appropriate acid in the presence of a solvent. The equivalent of the appropriate acid may vary between 0.5 and 1 mole equivalents. The reaction may be performed in a solvent, suitable solvents are ethers such is as 1,4-dioxane, diethyl ether or alcohols such as methanol, ethanol, propanol, or ketones such as acetone, isobutylmethylketone, or acetates such as ethyl acetate, butylacetate, or organic acids such as acetic acid, or water, or mixtures thereof. The total volume of solvents used may vary between 1 (v/w) to 100 (v/w) volume parts per weight of starting material, preferably between 10 (v/w) and 45 (v/w) volume parts per weight of starting material. The temperature of the reaction may be between −30 and 150° C., preferably between −5° C. and 100° C.

Pure compound of formula (I), 2-hydroxy-3-[5-(morpholin-4-ylmethyl)pyridin-2-yl]1H-indole-5-carbonitrile mesylate, esylate, edisylate, phosphate, fumarate or maleate, may be obtained by crystallising with or without an additive in suitable solvents to obtain a crystalline solid having a purity of about 95% and preferably about 98%

Another object of the present invention is the process for salt formation as described above.

### WORKING EXAMPLE

The following examples will describe, but not limit, the invention.

### Example 1

### 2-Hydroxy-3-[5-(morpholin-4-ylmethyl)pyridin-2-yl]1H-indole-5-carbonitrile mesylate

2-hydroxy-3-[5-(morpholin-4-ylmethyl)pyridin-2-yl]1H-indole-5-carbonitrile (0.6145 g) was suspended in ethanol (6.1 ml) and methane sulfonic acid (0.11 ml). The solution was heated to 40° C. for 14 hrs, and is then cooled to room temperature in 2 hrs. The crystals were filtered, washed with ethanol (2 ml) and dried in vacuum at 40° C. for 47 hrs. 0.6690 g 2-hydroxy-3-[5-(morpholin-4-ylmethyl)pyridin-2-yl]1H-indole-5-carbonitrile mesylate was obtained after drying.

¹H (500 MHz, DMSO-d₆): 14.75 (1H, br s), 10.97 (1H, s), 9.92 (1H, br s), 8.25 (1H, s), 8.00 (1H, s), 7.89 (2H, br m), 7.33 (1H, dd), 7.03 (1H, d), 4.30 (2H, br s), 3.98 (2H, br s), 3.66 (2H, br s), 3.37 (2H, br s), 3.12 (2H, br s), 2.38 (3H, s).

The crystals were analysed by X-ray powder diffraction (XRPD). The diffractogram shows the following d-values (given in angstrom) and relative intensities: 12.7(vs), 9.0 (w), 7.3 (vw), 6.4 (s), 5.8 (vw), 5.7 (vw), 5.3 (w), 4.93 (w), 4.80 (m), 4.72 (m), 4.53(m), 4.46 (m), 4.31 (m), 4.24 (m), 4.09 (s), 3.96 (m), 3.79 (m), 3.33 (s), 3.21 (m), 2.94 (w), 2.73 (w), 2.47 (w) Å.

The significant d-values (given in angstrom) and relative intensities are: 12.7(vs), 6.4 (s), 4.53 (m), 4.09 (s), 3.33 (s) Å.

The relative intensities (% rel.int.) are less reliable and instead of numerical values the following definitions are used:

vs (very strong): >83
s (strong): 60-83
m (medium): 17-60
w (weak): 7-17
vw (very weak): <7

### Example 2

### 2-Hydroxy-3-[5-(morpholin-4-ylmethyl)pyridin-2-yl]1H-indole-5-carbonitrile esylate

2-hydroxy-3-[5-(morpholin-4-ylmethyl)pyridin-2-yl]1H-indole-5-carbonitrile (0.5006 g) was suspended in ethanol (5 ml) and ethane sulfonic acid (0.12 ml). The solution was heated to 40° C. for 14 hrs, and was then cooled to room temperature in 2 hrs. The crystals were filtered, washed with ethanol (2 ml) and dried in vacuum at 40° C. for 47 hrs. 0.5487 g 2-hydroxy-3-[5-(morpholin-4-ylmethyl)pyridin-2-yl]1H-indole-5-carbonitrile esylate was to obtained after drying.

¹H (500 MHz, DMSO-d₆): 14.72 (1H, br s), 10.98 (1H, s), 9.85 (1H, br s), 8.25 (1H, s), 8.00 (1H, s), 7.88 (2H, dd), 7.34 (1H, d), 7.03 (1H, d), 4.32 (2H, br s), 4.00 (2H, br d), 3.64 (2H, br t), 3.37 (2H, br s), 3.13 (2H, bd s), 2.40 (2H, q), 1.07 (3H, t).

The crystals were analysed by X-ray powder diffraction (XRPD). The diffractogram shows the following d-values (given in angstrom) and relative intensities: 15.3(m), 12.6 (s), 9.1 (m), 7.6 (vw), 7.4 (m), 6.3 (m), 5.9 (w), 5.5 (w), 5.2 (vw), 5.0 (w), 4.87 (m), 4.74 (m), 4.47 (m), 4.32 (m), 4.16 (s), 4.12 (s), 4.04 (m), 3.85 (m), 3.41 (s), 3.19 (m), 2.92 (vw), 2.72 (w) Å.

The significant d-values (given in angstrom) and relative intensities are: 12.6(s), 6.3 (m), 4.47 (m), 4.16 (s), 4.12 (s), 3.41 (s) Å.

The relative intensities (% rel.int.) are less reliable and instead of numerical values the following definitions are used:

s (strong): >50
m (medium): 12-50
w (weak): 8-12
vw (very weak <8:

### Example 3

### 2-Hydroxy-3-[5-(morpholin-4-ylmethyl)pyridin-2-yl]1H-indole-5-carbonitrile edisylate

2-hydroxy-3-[5-(morpholin-4-ylmethyl)pyridin-2-yl]1H-indole-5-carbonitrile (0.6568 g) was suspended in ethanol (6.55 ml) and 1,2-ethane disulfonic acid (0.3471 g. The solution was heated to 40° C. for 14 hrs, and was then cooled to room temperature in 2 hrs. The crystals were filtered, washed with ethanol (2 ml) and dried in vacuum at 40° C. for 47 hrs. 0.7797 g 2-hydroxy-3-[5-(morpholin-4-ylmethyl)pyridin-2-yl]1H-indole-5-carbonitrile edisylate was obtained after drying.

¹H (500 MHz, DMSO-d₆): 14.74 (1H, br s), 10.98 (1H, s), 9.87 (1H, br s), 8.27 (1H, s), to 8.00 (1H, d), 7.89 (2H, br m), 7.33 (1H, dd), 7.03 (1H, d), 4.33 (2H, s), 4.00 (2H, d), 3.65 (2H, t), 3.39 (2H, d), 3.14 (2H, br q), 2.74 (4H, s).

The crystals were analysed by X-ray powder diffraction (XRPD). The diffractogram shows the following d-values (given in angstrom) and relative intensities: 19.9(m), 18.1 (w), 15.3(s), 13.2 (vw), 11.3 (m), 9.0 (vw), 8.2 (w), 8.0 (w), 7.6 (w), 7.4 (w), 6.8 (w), 6.6 (m), 6.3 (m), 6.0 (w), 5.6 (s), 5.4 (m), 5.2 (m), 5.1 (m), 5.0 (m), 4.85 (m), 4.32 (s), 4.24 (m), 4.17 (w), 4.12(s), 4.08 (s), 3.90 (w), 3.82 (w), 3.66 (w), 3.53 (m), 3.35 (m), 3.27 (m), 3.00 (vw) Å.

The significant d-values (given in angstrom) and relative intensities are: 15.3(s), 11.3 (m), 5.6 (s), 4.32 (s), 4.12 (s), 4.08 (s) Å.

The relative intensities (% rel.int.) are less reliable and instead of numerical values the following definitions are used:

s (strong): >55
m (medium): 24-55
w (weak): 13-24
vw (very weak): <13

### Example 4

### 2-hydroxy-3-[5-(morpholin-4-ylmethyl)pyridin-2-yl]1H-indole-5-carbonitrile phosphate

2-hydroxy-3-[5-(morpholin-4-ylmethyl)pyridin-2-yl]1H-indole-5-carbonitrile (0.5919 g) was suspended in ethanol (5.9 ml) and o-phosphorous acid (0.1 ml. The solution was heated to 40° C. for 14 hrs, and was then cooled to room temperature in 2 hrs. The crystals were filtered, washed with ethanol (2 ml) and dried in vacuum at 40° C. for 47 hrs. 0.6666 g 2-hydroxy-3-[5-(morpholin-4-ylmethyl)pyridin-2-yl]1H-indole-5-carbonitrile phosphate was obtained after drying.

¹H (500 MHz, DMSO-d₆): 14.78 (1H, br s), 10.90 (1H, s), 8.12 (1H, s), 7.91 (1H, s), 7.85 (1H, br d), 7.81 (1H, dd), 7.29 (1H, dd), 7.01 (1H, d), 3.60 (4H, br t), 3.46 (2H, s), 2.45 (4H, br s).

The crystals were analysed by X-ray powder diffraction (XRPD). The diffractogram shows the following d-values (given in angstrom) and relative intensities: 15.2(m), 7.6 (w), 5.1 (w), 4.12 (vw), 2.29 (vw) Å.

The significant d-values (given in angstrom) and relative intensities are: 15.2(m), 7.6 (w), 5.1 (w), 4.12 (vw) Å.

The relative intensities (% rel.int.) are less reliable and instead of numerical values the following definitions are used:

m (medium): >50
w (weak): 15-50
vw (very weak):<15

### Example 5

### 2-hydroxy-3-[5-(morpholin-4-ylmethyl)pyridin-2-yl]1H-indole-5-carbonitrile fumarate

2-hydroxy-3-[5-(morpholin-4-ylmethyl)pyridin-2-yl]1H-indole-5-carbonitrile (0.5765 g) was suspended in ethanol (5.7 ml) and fumaric acid (0.1893 g). The solution was heated to 40° C. for 14 hrs, and was then cooled to room temperature in 2 hrs. The crystals were filtered, washed with ethanol (2 ml) and dried in vacuum at 40° C. for 47 hrs. 0.6316 g 2-hydroxy-3-[5-(morpholin-4-ylmethyl)pyridin-2-yl]1H-indole-5-carbonitrile fumarate was obtained after drying.

¹H (500 MHz, DMSO-d₆): 14.78 (1H, br s), 10.89 (1H, s), 8.11 (1H, br s), 7.91 (1H, br s), 7.85 (1H, br d), 7.80 (1H, br dd), 7.28 (1H, dd), 7.00 (1H, d), 6.63 (2H, s), 3.59 (4H, br t), 3.42 (2H, s), 2.42 (4H, br s).

The crystals were analysed by X-ray powder diffraction (XRPD). The diffractogram shows the following d-values (given in angstrom) and relative intensities: 19.8(w), 16.5 (m), 15.3 (w), 13.1 (m), 12.7 (m), 12.0 (w), 10.5 (w), 9.1 (w), 7.6 (w), 6.8 (m), 6.5 (w), 6.3 (w), 6.2(w), 6.0 (m), 5.6 (w), 5.5 (w), 5.2 (w), 5.1 (w), 4.94 (w), 4.86 (m), 4.71 (w), 4.63 (w), 4.55 (m), 4.38 (m), 4.23 (w), 4.12 (w), 3.58 (m), 3.40 (m), 3.26 (s), 3.12 (m) Å.

The significant d-values (given in angstrom) and relative intensities are: 16.5(m), 13.1 (m), 12.7 (m), 6.8 (m), 3.26 (s) Å.

The relative intensities (% rel.int.) are less reliable and instead of numerical values the following definitions are used:

s (strong): >75
m (medium): 33-75
w (weak): <33

### Example 6

### 2-hydroxy-3-[5-(morpholin-4-ylmethyl)pyridin-2-yl]1H-indole-5-carbonitrile maleate

2-hydroxy-3-[5-(morpholin-4-ylmethyl)pyridin-2-yl]1H-indole-5-carbonitrile (0.5473 g) was suspended in ethanol (5.47 ml) and maleic acid (0.1719 g). The solution was heated to 40° C./14 hrs, and was then cooled to room temperature in 2 hrs. The crystals were filtered, washed with ethanol (2 ml) and dried in vacuum at 40° C. for 47 hrs. 0.5902 g 2-hydroxy-3-[5-(morpholin-4-ylmethyl)pyridin-2-yl]1H-indole-5-carbonitrile phosphate was obtained after drying.

¹H (500 MHz, DMSO-d₆): 14.75 (1H, br s), 10.94 (1H, s), 8.19 (1H, s), 7.96 (1H, s), 7.88 (1H, br d), 7.83 (1H, dd), 7.32 (1H, dd), 7.02 (1H, d), 6.11 (2H, s), 3.93 (2H, br s), 3.72 (4H, br s), 2.89 (4H, br s).

The crystals were analysed by X-ray powder diffraction (XRPD). The diffractogram shows the following d-values (given in angstrom) and relative intensities: 20.1(w), 18.5 (m), 15.3 (w), 12.7 (s), 10.3 (m), 10.0 (m), 9.0 (m), 7.9 (vw), 7.6 (w), 7.4 (w), 6.8 (s), 6.5 (w), 6.3 (m), 6.1 (m), 5.6 (w), 5.3 (w), 5.2 (w), 4.78 (m), 4.67 (m), 4.58 (s), 4.46 (m), 4.36 (m), 4.23 (m), 3.98 (w), 3.79 (m), 3.14 (w), 3.05 (m), 2.94 (w) A.

The significant d-values (given in angstrom) and relative intensities are: 12.7(s), 6.8 (s), 6.1 (m), 4.58 (s), 3.05 (m) Å.

The relative intensities (% rel.int.) are less reliable and instead of numerical values the following definitions are used:

s (strong): >50
m (medium): 14-50
w (weak): 4-14
vw (very weak): <4

Crystalline 2-hydroxy-3-[5-(morpholin-4-ylmethyl)pyridin-2-yl]1H-indole-5-carbonitrile salts were analyzed using X-ray powder diffraction (XRPD) as described below:

The peaks, identified with d-values calculated from the Bragg formula and intensities, have been extracted from the diffractogram of crystalline salt. Only the main peaks, that are the most characteristic, significant, distinct and/or reproducible, have been tabulated, but additional peaks can be extracted, using conventional methods, from the diffractogram. The presence of these main peaks, reproducible and within the error limit, is for most circumstances sufficient to establish the presence of said crystalline salt.

X-ray diffraction analyses were performed using a PANalytical X'Pert Pro MPD diffractometer for 64 minutes from 1 to 40° 20 with and without internal standard reference. The 20 angles were corrected with regard to the standard values whereafter calculation into d-values (distance values) was done. The d-values may vary in the range ±2 on the last given decimal place. The sample preparation was performed according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London or Klug, H. P. & Alexander, L. E. (1974), X-ray Diffraction Procedures, John Wiley and Sons, New York.

### Pharmacology

Determination of ATP competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK313 (Dundee University, UK), 12 mM morpholinepropanesulfonic acid (MOPS), pH 7.0, 03 mM EDTA, 0.01% β-mercaptorethanol, 0.004% Brij 35 (a natural detergent), 0.5% glycerol and 0.5 μg BSA/25 The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (KO of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

The Kᵢ value for the new salts, the mesylate, esylate, edisylate, phosphate, fumarate and maleate of 2-hydroxy-3-[5-(morpholin-4-ylmethyl)pyridin-2-yl]1H-indole-5-carbonitrile of the present invention are in the range of 0.001 nM to 300 nM.

### Hygroscopicity

### Dynamic Vapour Sorption Analysis (DVS)

The studies were undertaken using Dynamic Vapour Sorption Appararatus (DVS, Surface Measurement Systems, London UK). The apparatus consists of Cahn micobalance housed inside a temperature-controled cabinet. All experiments were performed at 25° C. The DVS was used to characterize the moisture uptake (% w/w) at different relative humidities (RH). Samples (5-10 mg) were weighed directly into the DSV sample cup and exposed to different relative humidities.

### Results

| | Relative humidities (RH) | | |
|---|---|---|---|
| Sample | 40% | 60% | 80% |
| HCl | 8.7 | 11.5 | 13.0 |
| Mesylate | 0.7 | 1.1 | 1.6 |
| Esylate | 0.5 | 0.8 | 1.2 |
| Edisylate | 5.1 | 6.6 | 8.2 |
| Phosphate | 4.1 | 6.6 | 10.8 |
| Fumarate | 3.8 | 4.5 | 5.1 |
| Maleate | 1.7 | 2.2 | 2.7 |

It is clear from the results above that the mesylate, esylate, edisylate, phosphate, fumarate and maleate salts of 2-hydroxy-3-[5-(morpholin-4-ylmethyl)pyridin-2-yl]1H-indole-5-carbonitrile show a much lower hygroscopicity than the hydrochloride salt thereof and are thus more suitable for preparing pharmaceutical formulations.

1. A pharmaceutically acceptable salt of the compound of formula (I) 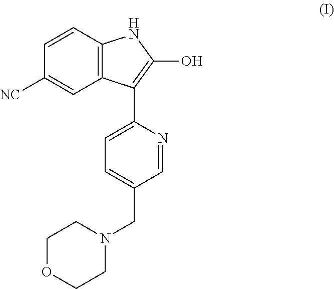
which is a mesylate, esylate, edisylate, phosphate, fumarate or maleate salt.

2. The pharmaceutically acceptable salt according to claim 1 which is 2-hydroxy-3-[5-(morpholin-4-ylmethyl)pyridin-2-yl]1H-indole-5-carbonitrile mesylate.

3. The salt according to claim 2, characterized by the X-ray powder diffraction d-values and relative intensities 12.7(vs), 6.4 (s), 4.53 (m), 4.09 (s) and 3.33(s) Å.

4. The salt according to claim 1 in a substantially crystalline form.

5. The pharmaceutically acceptable salt according to claim 1 which is 2-hydroxy-3-[5-(morpholin-4-ylmethyl)pyridin-2-yl]1H-indole-5-carbonitrile esylate.

6. The salt according to claim 5, characterized by the X-ray powder diffraction d-values and relative intensities 12.6(s), 6.3 (m), 4.47 (m), 4.16 (s), 4.12 (s) and 3.41(s) Å.

7. (canceled)

8. The pharmaceutically acceptable salt according to claim 1 which is 2-hydroxy-3-[5-(morpholin-4-ylmethyl)pyridin-2-yl]1H-indole-5-carbonitrile edisylate.

9. The salt according to claim 8, characterized by the X-ray powder diffraction d-values and relative intensities 15.3(s), 11.3 (m), 5.6 (s), 4.32 (s), 4.12 (s) and 4.08 (s) Å.

10. (canceled)

11. The pharmaceutically acceptable salt according to claim 1 which is 2-hydroxy-3-[5-(morpholin-4-ylmethyl)pyridin-2-yl]1H-indole-5-carbonitrile phosphate.

12. The salt according to claim 11, characterized by the X-ray powder diffraction d-values and relative intensities 15.2(m), 7.6 (w), 5.1 (w) and 4.12(vw) Å.

13. (canceled)

14. The pharmaceutically acceptable salt according to claim 1 which is 2-hydroxy-3-[5-(morpholin-4-ylmethyl)pyridin-2-yl]1H-indole-5-carbonitrile fumarate.

15. The salt according to claim 14, characterized by the X-ray powder diffraction d-values and relative intensities 16.5(m), 13.1 (m), 12.7 (m), 6.8 (m) and 3.26(s) Å.

16. (canceled)

17. The pharmaceutically acceptable salt according to claim 1 which is 2-hydroxy-3-[5-(morpholin-4-ylmethyl)pyridin-2-yl]1H-indole-5-carbonitrile maleate.

18. The salt according to claim 17, characterized by the X-ray powder diffraction d-values and relative intensities 12.7(s), 6.8 (s), 6.1 (m), 4.58 (s) and 3.05(m) Å.

19-29. (canceled)

30. A pharmaceutical formulation comprising as an active ingredient a therapeutically effective amount of a salt according to claim 1, optionally in association with at least one diluent, excipient or inert carrier.

31-52. (canceled)

53. A method of prevention and/or treatment of conditions associated with glycogen synthase kinase-3, comprising administering to a mammal, including man in need of such prevention and/or treatment, a therapeutically effective amount of a salt as defined in claim 1.

54. A method of prevention and/or treatment of cognitive disorders, comprising administering to a mammal, including man in need of such prevention and/or treatment, a therapeutically effective amount of a salt as defined in claim 1 wherein the cognitive disorder is selected from: dementia, Cognitive Deficit in Schizophrenia (CDS), Mild Cognitive Impairment (MCI), Age-Associated Memory Impairment (AAMI), Age-Related Cognitive Decline (ARCD) or Cognitive Impairment No Dementia (CIND), and wherein dementia is associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration, traumatic brain injury (TBI) or dementia pugilistica, Alzheimer's Disease (AD), Down syndrome, vascular dementia, Parkinson's Disease (PD), postencephalitic parkinsonism, dementia with Lewy bodies, HIV dementia, Huntington's Disease, amyotrophic lateral sclerosis (ALS), motor neuron diseases (MND), Creurtfeld-Jacob's disease or prion diseases or treatment for the delay of disease progression of Alzheimer's Disease.

55-61. (canceled)

62. A method of prevention and/or treatment of Bipolar Disorder including acute mania, bipolar depression, bipolar maintenance, major depressive disorders (MDD) including depression, major depression, mood stabilization, schizoaffective disorders including schizophrenia, dysthymia, attention deficit disorder (ADD), attention deficit hyperactivity disorder (ADHD) or affective disorders, comprising administering to a mammal, including man in need of such prevention and/or treatment, a therapeutically effective amount of a salt as defined in claim 1.

63. (canceled)

64. A method of prevention and/or treatment of Type I diabetes, Type II diabetes, diabetic neuropathy, alopecia or inflammatory diseases, bone related disorders or conditions, osteoporosis, comprising administering to a mammal, including man in need of such prevention and/or treatment, a therapeutically effective amount of a salt as defined in claim 1.

65-66. (canceled)

67. A method of increasing bone formation, cancellous bone formation, increasing bone mineral density or reducing the incidence of fractures, or enhancing fracture healing, comprising administering to a mammal, in need of such prevention and/or treatment, a therapeutically effective amount of a compound as described in claim 1.

68-73. (canceled)
